(19) United States
(12) Patent Application Publication
(10) Pub. No.: US 2019/0183389 A1
(43) Pub. Date: Jun. 20, 2019

(54) Activity Detection Device, Activity Detection System And Activity Detection Method

(71) Applicant: CASIO COMPUTER CO., LTD., Tokyo (JP)

(72) Inventor: Taisuke Koroku, Sagamihara-shi (JP)

(21) Appl. No.: 16/221,056

(22) Filed: Dec. 14, 2018

(30) Foreign Application Priority Data
Dec. 20, 2017 (JP) 2017-243375

(51) Int. Cl. A61B 5/11

(52) U.S. Cl. CPC A61B 5/1123; A61B 5/1126; A61B 5/1118

(57) ABSTRACT

An object is to appropriately detect various activities of a detection target. An electronic device transmits information characterizing the activity of a detection target, and an activity detection device acquires, from its sensor section, a sensing motion signal that is motion information which successively fluctuates in accordance with motions of the detection target as first information. The activity detection device also acquires, from the electronic device, the information characterizing the activity of the detection target as second information, and identifies the type of the activity of the detection target by using the acquired first and second information.

### CROSS-REFERENCE TO RELATED APPLICATION

This application is based upon and claims the benefit of priority from the prior Japanese Patent Application No. 2017-243375, filed Dec. 20, 2017, the entire contents of which are incorporated herein by reference.

### BACKGROUND OF THE INVENTION

### 1. Field of the Invention

The present invention relates to an activity detection device, an activity detection system and an activity detection method for detecting the activity of a detection target.

### 2. Description of the Related Art

Generally, in fields, such as a sport or a lifelog, activity detection devices are utilized for exercise management or life management. In these activity detection devices, for example, the motion of a detection target is detected by an acceleration sensor or a gyro sensor, and the detected motion signal is analyzed so as to find and store an exercise state, the amount of exercise, consumed energy and the like. As this type of activity detection device, a general-purpose detection device which can detect, by this single device alone, various activities such as walking, jogging, toothbrushing, desk work and the like has been conventionally disclosed in, for example, Japanese Patent Application Laid-Open (Kokai) Publication No. 2012-000211.

### SUMMARY OF THE INVENTION

In accordance with one aspect of the present invention, there is provided an activity detection device comprising: a memory; and a CPU, wherein the CPU performs, based on a program stored in the memory, processing including: acquiring motion information that successively fluctuates in accordance with motions of a detection target, as first information; acquiring information characterizing activity of the detection target, as second information; and identifying a type of the activity of the detection target by using the first information and the second information.

In accordance with another aspect of the present invention, there is provided an activity detection device comprising: a memory; and a CPU, wherein the CPU performs, based on a program stored in the memory, processing including: acquiring motion information that successively fluctuates in accordance with motions of a detection target, as first information; acquiring information whose type is different from a type of the motion information and which is characterizing activity of the detection target, as second information; identifying a type of the activity of the detection target by using the second information; and performing analysis corresponding to the identified type of the activity of the detection target on the first information.

In accordance with another aspect of the present invention, there is provided an activity detection system constituted by an activity detection device that detects activity of a detection target and an electronic device being communicably connected to each other, wherein the electronic device comprises a memory and a CPU which performs, based on a program stored in the memory, processing of transmitting information characterizing the activity of the detection target, and wherein the activity detection device comprises a memory and a CPU which performs, based on a program stored in the memory, processing of (i) acquiring motion information that successively fluctuates in accordance with motions of the detection target, as first information, (ii) acquiring information characterizing the activity of the detection target, as second information, and (iii) identifying a type of the activity of the detection target by using the first information and the second information.

In accordance with another aspect of the present invention, there is provided an activity detection method for an activity detection device comprising: acquiring motion information that successively fluctuates in accordance with motions of a detection target, as first information; acquiring information characterizing activity of the detection target, as second information; and identifying a type of the activity of the detection target by using the first information and the second information.

The above and further objects and novel features of the present invention will more fully appear from the following detailed description when the same is read in conjunction with the accompanying drawings. It is to be expressly understood, however, that the drawings are for the purpose of illustration only and are not intended as a definition of the limits of the invention.

### BRIEF DESCRIPTION OF THE DRAWINGS

FIG. 1 is a diagram showing an activity detection system constituted by an electronic device 1 on a treadmill side being communicably connected to an activity detection device 2 on the side of a detection target using the treadmill;

FIG. 2 is a diagram showing an activity detection system constituted by the electronic device 1 on a bicycle side being communicably connected to the activity detection device 2 on the side of a detection target using the bicycle;

FIG. 3 is a block diagram showing basic components of the electronic device 1;

FIG. 4 is a block diagram showing basic components of the activity detection device 2;

FIG. 5 is a flowchart outlining the operation of the electronic device 1;

FIG. 6 is a flowchart outlining the operation of the activity detection device 2;

FIG. 7 is a flowchart outlining the operation of the activity detection device 2 in a first modification example of a first embodiment;

FIG. 8 is a block diagram showing basic components of the activity detection device 2 in a second embodiment; and

FIG. 9 is a flowchart outlining the operation of the activity detection device 2 in the second embodiment.

### DETAILED DESCRIPTION OF THE PREFERRED EMBODIMENTS

Embodiments of the present invention will hereinafter be described in detail with reference to the drawings.

### First Embodiment

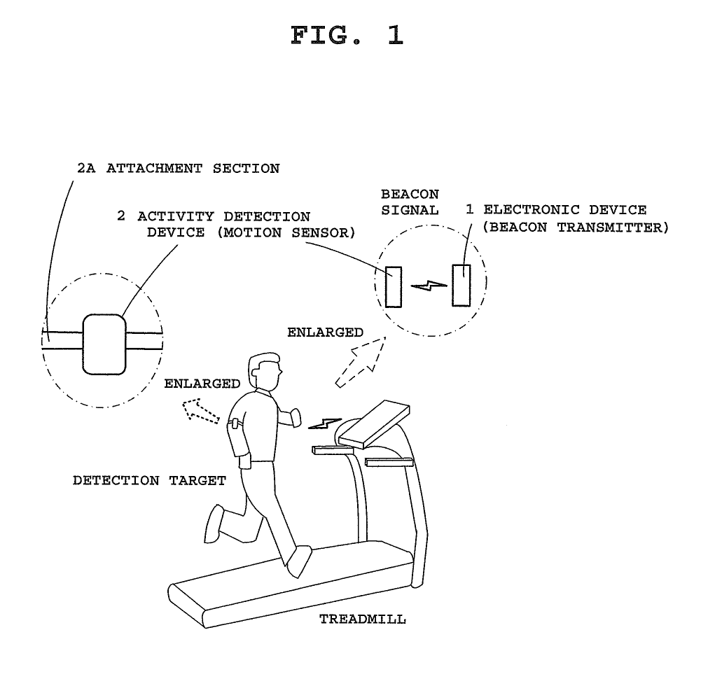

First, a first embodiment of the present invention is described with reference to FIG. 1 to FIG. 6.

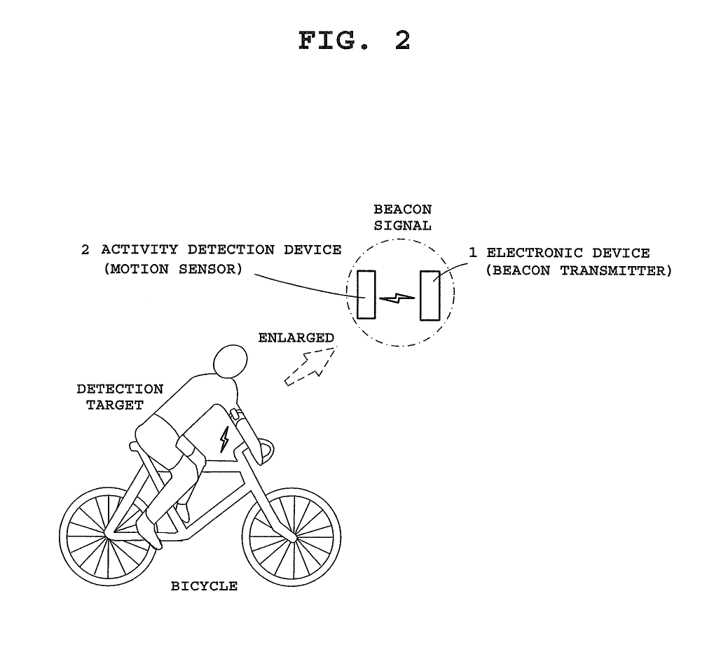

FIG. 1 and FIG. 2 are diagrams each showing an activity detection system constituted by an electronic device 1 and an activity detection device 2 being communicably connected. FIG. 1 shows a state in which a detection target (for example, user) is performing activity (jogging) using an activity device (treadmill), and FIG. 2 shows a state in which a detection target is performing activity (cycling) using an activity device (bicycle).

The electronic device 1 and the activity detection device 2 constituting this activity detection system can be linked to each other (they can transmit or receive data to and from each other) via short-distance wireless communication. Note that “dotted arrows” and “enlarged” in the drawings indicate an enlarged display of the electronic device 1 and the activity detection device 2.

The electronic device 1 is a small-sized device provided on the side of a use object such as a treadmill or bicycle that is being used by a detection target. In the present embodiment, the electronic device 1 can be removably attached to various use objects, such as a bicycle or treadmill, for general purposes. Note that the electronic device 1 may be fixedly attached to an activity device at the time of shipping, or may be installed near a use object without being attached thereto.

The “use object” refers to an object that is used by a detection target at the time of activity, and includes, for example, a treadmill, a bicycle, bedding, a writing instrument and a toothbrush instrument as well as a train, an automobile and a bus. That is, the “use object” is not limited to an activity device or daily lifestyle instrument but may be a vehicle. The electronic device 1 is a beacon transmitter configured to have a short-distance wireless communication function (BLE: Bluetooth Low Energy) in Bluetooth (registered trademark) standards and transmit a beacon signal by this communication function. This beacon signal includes information (characterization activity information) characterizing the activity of a detection target, and is continuously outputted and transmitted.

The “characterization activity information” refers to information (use object ID) for identifying the type of a use object such as an activity device (such as treadmill or bicycle) being used by a detection target, and any “characterization activity information” can be set by a user operation. Note that the “characterization activity information” is not limited to the above-described use object ID, and may be information (activity type ID) for identifying activity, such as walking or jogging when the user is using a treadmill, cycling when the user is using a bicycle, or automobile driving when the user is driving an automobile. Moreover, the “characterization activity information” is not limited to the above-described use object ID or activity type ID, and may be information that implies or indicates activity.

The activity detection device 2 is a device for detecting motion information (sensing motion signal) of a detection target which successively fluctuates in accordance with motions of the detection target, and can be removably attached to the detection target. That is, the activity detection device 2 is a small-sized wearable motion sensor (sensing terminal) having various sensors (omitted in FIG. 1) for detecting motions of a detection target. This activity detection device 2 is provided with an attachment section 2A that can be attached to any part of a detection target (upper arm in the example of the drawing). Note that, although this attachment section 2A has a simple structure where a belt is used to fasten the device to an upper arm, the present invention is not limited thereto, and an arbitrary structure may be adopted, such a structure using a clip.

Also, the activity detection device 2 has a short-distance wireless communication function so as to receive, from the external electronic device 1, a beacon signal having information characterizing the activity of a detection target. When the beacon signal is received from the external electronic device 1, the activity detection device 2 performs processing of identifying the type of the activity of the detection target by using a sensing motion signal (motion information: first information) detected by the activity detection device 2 and the received beacon signal (information characterizing the activity of the detection target: second information). Here, “using the first information and the second information” is not limited to “using the first information and the second information simultaneously in time”, and there are specifications where the first information and the second information are used simultaneously and specifications where the first information and the second information are used not simultaneously (for example, specifications in which a mode of using the first information and a mode of using the second information are provided and switching can be made between these modes).

When the type of the activity of a detection target, such as walking or jogging when the detection target is using a treadmill, cycling when the detection target is using a bicycle, or automobile driving when the detection target is using an automobile, is identified, the activity detection device 2 selects an activity analysis algorithm corresponding to the identified activity type from among a plurality of activity analysis algorithms prepared corresponding to plural types of activities in advance, analyzes motion information (sensing motion signal) of the detection target by following the selected activity analysis algorithm, and stores the analysis result.

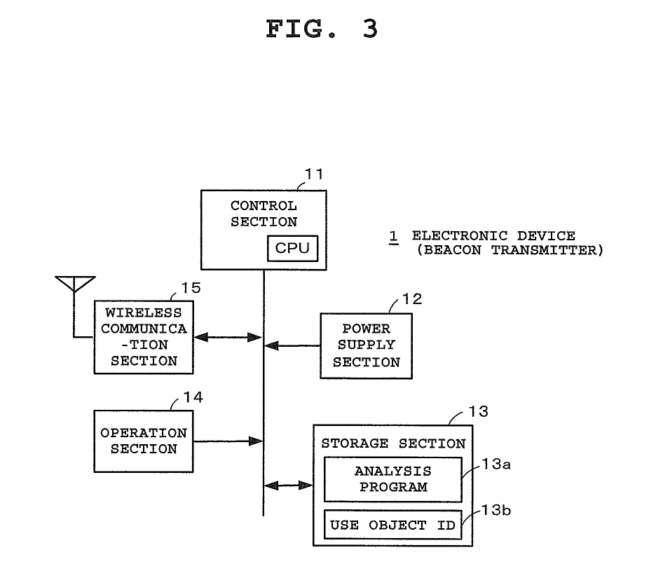

FIG. 3 is a block diagram showing basic components of the electronic device (beacon transmitter) 1.

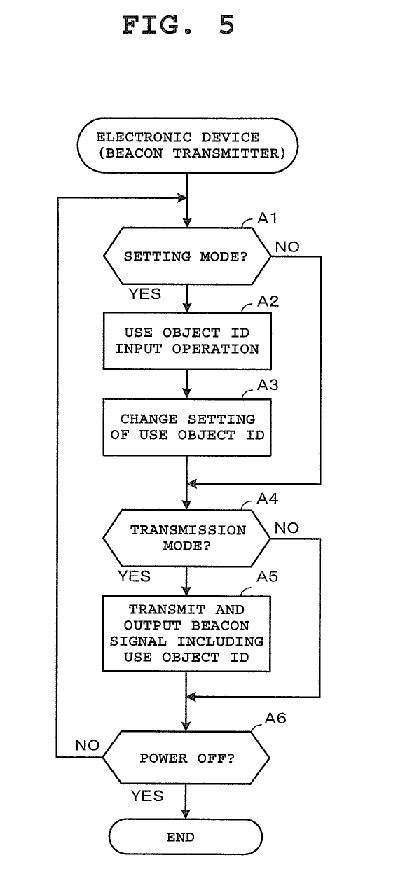

The electronic device 1 has a control section 11 as its centerpiece. The control section 11 operates by power supply from a power supply section (secondary battery) 12, and a CPU (central processing unit) of this control section 11 controls the entire operation of the electronic device 1 in accordance with various programs stored in a storage section 13. In the control section 11, a memory and the like are also provided. The storage section 13 includes a ROM (Read-Only Memory), a flash memory and the like, and has stored therein programs and various applications for achieving the first embodiment in accordance with an operation procedure shown in FIG. 5. In the present embodiment, the storage section 13 has stored therein an analysis program 13a where a plurality of activity analysis algorithms prepared in advance have been written corresponding to plural types of activities as described above. In addition, the storage section 13 has stored therein information (use object ID) 13b for identifying, when a detection target is performing activity by using an activity device or the like, the type of this use object.

To the control section 11, an operation section 14 and a wireless communication section 15 are connected as its input/output devices. Although not shown, the operation section 14 has a power supply key for powering ON/OFF, a mode key for switching between a setting mode and a transmission mode, a key for inputting the above-described use object ID and the like. Here, the setting mode is an operation mode for inputting and setting a use object ID, and the transmission mode is an operation mode for transmitting and outputting this set use object ID. The wireless communication section 15 continuously transmits and outputs a beacon signal with a use object ID included in an advertising packet of the beacon signal based on BLE. The radio wave reachable range of this advertising packet is set within a range of about 30 cm for the electronic device 1 provided to a treadmill/bicycle side and a range of about 2 m for the electronic device 1 provided to an automobile. That is, this first embodiment is configured such that, by the radio wave intensity of the advertising packet being changed in accordance with an activity device type, the effective range (radio wave reachable range) of the beacon is varied (interference is prevented).

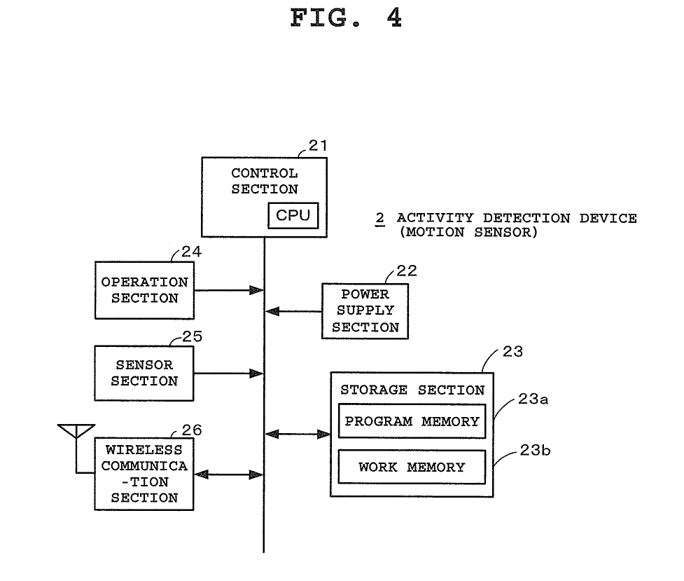

FIG. 4 is a block diagram showing basic components of the activity detection device 2.

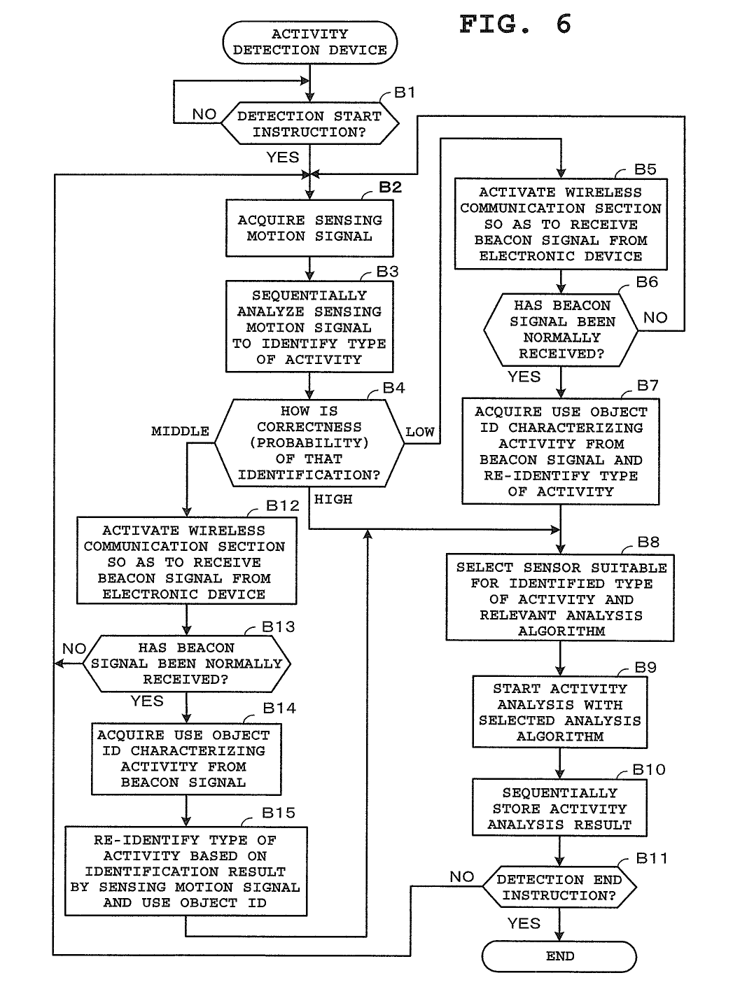

The activity detection device 2 is structured with a control section 21 as its centerpiece. This control section 21 operates by power supply from a power supply section (secondary battery) 22, and a CPU (central processing unit) of this control section 21 controls the entire operation of this activity detection device 2 in accordance with various programs stored in a storage section 23. This control section 21 is provided with not only the CPU but also with a memory and the like. The storage section 23 is structured to include a ROM, a flash memory and the like, and has a program memory 23a where programs and various applications for achieving the first embodiment in accordance with an operation procedure shown in FIG. 6 are stored, a work memory 23b where data such as a flag is temporarily stored and the like. Note that this storage section 23 may be structured to include a removable portable memory (recording medium) such as an SD (Secure Digital) card or a USB (Universal Serial Bus) memory, or may be structured to include a storage area on a predetermined server apparatus side in a case where the activity detection device 2 is connected to a network by a communication function.

To the control section 21, an operation section 24, a sensor section 25, and a wireless communication section 26 are connected as its input devices. Although not shown, the operation section 24 has a power supply key for powering ON/OFF, a start/end key for giving an instruction for starting or ending detection and the like. The control section 21 performs processing in accordance with an input operation signal from this operation section 24. The sensor section 25 has various sensors such as a triaxial acceleration sensor, a gyro sensor, and a geomagnetic sensor (omitted in the drawing) so as to detect acceleration, inclination, direction and the like. This sensor section 25 is a three-dimensional motion sensor (motion detection section) which detects various motions such as slow motions and quick motions by taking advantage of the characteristics of the sensors, and outputs a sensing motion signal that successively fluctuates in accordance with motions of a detection target. The activity detection device 2 has a thin and rectangular housing, and detects a motion with the direction of the short side of the housing as an X-axis direction, the direction of the long side of the housing as a Y direction, and the thickness direction of the housing as a Z direction in a triaxial system. Note that the various sensors constituting the sensor section (motion detection section) 25 are not limited to triaxial sensors.

The wireless communication section 26 actualizes a short-distance wireless communication function (BLE) of Bluetooth (registered trademark) standards. The control section 21 analyzes a sensing motion signal that is successively fluctuating in accordance with motions of a detection target and, if necessary, activates this wireless communication section 26. When a beacon signal that is being continuously transmitted from the electronic device 1 is received with the wireless communication section 26 being in the activated state, the control section 21 acquires a use object ID in the advertising packet of the beacon signal, and performs processing of identifying the type of the activity of the detection target by using this use object ID.

Next, the operation concept of the electronic device (beacon transmitter) 1 and the activity detection device 2 in the first embodiment is described with reference to flowcharts shown in FIG. 5 and FIG. 6. Here, each function described in these flowcharts is stored in a readable program code format, and operations based on these program codes are sequentially performed. Also, operations based on the above-described program codes transmitted over a transmission medium such as a network can also be sequentially performed. That is, the unique operations of the present embodiment can be performed using programs and data supplied from an outside source over a transmission medium, in addition to a recording medium. This applies to other embodiments described later.

FIG. 5 is a flowchart outlining the operation of the electronic device (beacon transmitter) 1.

First, the control section 11 (more specifically, the CPU of the of the control device 11) of the electronic device 1 judges whether the mode key (omitted in the drawing) of the operation section 14 has been operated to switch the current operation mode to the setting mode (Step A1) or to the transmission mode (Step A4). In the setting mode (YES at Step A1), when an operation of inputting a use object ID is performed (Step A2), the control section 11 performs processing of changing the setting by overwriting the storage section 13 with the inputted use object ID (Step A3). Note that, in order to decrease the number of times of key operations for inputting the use object ID, a configuration may be adopted in which the number of times the same key is successively pressed can be inputted as the use object ID.

Also, when the current operation mode is switched to the transmission mode (YES at Step A4), the control section 11 reads out the use object ID from the storage section 13, puts this use object ID into the advertising packet of a beacon signal, and transmits and outputs this beacon signal through the wireless communication section 26 (Step A5). Here, the control section 11 judges the type of the use object based on this use object ID, and transmits and outputs the beacon signal by changing the radio wave intensity (radio wave reachable range) of the advertising packet in accordance with the type. Next, the control section 11 returns to Step A6 and judges whether the power supply has been turned OFF. Then, the control section 11 returns to the above-described Step A1, and repeats the above-described operations until the power supply is turned OFF.

FIG. 6 is a flowchart outlining the operation of the activity detection device 2.

When an instruction for starting detection is given by an operation on the start/end key of the operation section 24 (YES at Step B1), the control section 21 (more specifically, the CPU of the control section 21) of the activity detection device 2 acquires a sensing motion signal (motion information) from the sensor section (motion detection section) 25 (Step B2). Also, by sequentially analyzing this sensing motion signal, the control section 21 provisionally identifies the type of the activity of the detection target (Step B3), and judges the correctness (probability) of the identification result (Step B4). That is, the control section 21 judges whether the activity of the detection target can be detected based on the sensing motion signal. Here, in order to provisionally judge the type of the activity of the detection target, the control section 21 judges whether the correctness (probability) of the identification result is lower than a first threshold (for example, 30%), higher than a second threshold (for example, 80%), or between the above-described first and second thresholds, that is, in the middle therebetween.

Here, when the correctness (probability) of the identification result is higher than the second threshold (for example, 80%), for example, when a certain characteristic conspicuously appears in the waveform of the motion signal and the motion signal is a signal having a characteristic waveform specific to jogging or the like in many respects such as an oscillation state (intensity and cycle), the control section judges that the correctness (probability) of the identification result is high. That is, the control section 21 judges that the activity of the detection target can be detected based on the sensing motion signal. In this case, the control section 21 takes the identification result (the type of the activity identified by using the sensing motion signal) at Step B3 described above as effective without using the above-described beacon signal (characterization activity information: second information) continuously transmitted from the electronic device 1, and then proceeds to Step B8 to Step B11.

That is, the control section 21 first selects a sensor suitable for the identification result (type of activity) from among various sensors in the sensor section (motion detection section) 25, and selects an activity analysis algorithm relevant to the identification result (type of activity) from among a plurality of activity analysis algorithms (Step B8). Then, the control section 21 starts this selected activity analysis algorithm to start activity analysis processing based on a sensing motion signal from the selected sensor (Step B9), and causes the analysis result to be sequentially stored in the storage section 23 (Step B10). Next, the control section 21 judges whether an instruction for ending the detection has been given by an operation on the start/end key of the operation section 24 (Step B11), and returns to the above-described Step B2 when an instruction for ending the detection has not been given. Then, while identifying the type of the activity of the detection target by using the sensing motion signal, the control section 21 judges the correctness of the identification result (Steps B3 and B4) as described above, and repeats the above-described operations as long as the correctness is high.

When the correctness (probability) of the identification result is lower than the first threshold (for example, 30%), for example, when the motion signal is a slow and less-characteristic waveform signal, the control section 21 judges that the correctness (probability) of the identification result is low. That is, the control section 21 judges that it is impossible to detect the activity of the detection target based on the sensing motion signal. In this case, the control section 21 proceeds to Step B5 and activates the wireless communication section 26 so as to receive the beacon signal continuously transmitted from the electronic device 1. Here, the control section 21 judges whether the activity detection device 2 has entered the radio wave reachable range of the beacon signal, that is, whether the beacon signal has been normally received (Step B6). Then, when the beacon signal has not been normally received (NO at Step B6), the control section 21 then returns to the above-described Step B2. Then, while identifying the type of the activity of the detection target by using the sensing motion signal, the control section 21 judges the correctness of the identification result (Steps B3 and B4) as described above, and repeats the above-described operations as long as the correctness is low.

During this repetition, when the beacon signal is normally received with the correctness (probability) of the identification result being kept low (YES at Step B6), the control section 21 acquires information (use object ID) characterizing the activity of the detection target from the advertising packet of the received beacon signal, and re-identifies the type of the activity of the detection target by using this use object ID (Step B7). The control section 21 then takes the re-identification result (type of activity) acquired at the above-described Step B7 as effective without using the identification result (type of activity) acquired at the above-described Step B3, and proceeds to perform the above-described Step B8 to Step B11. Then, the control section 21 performs processing of selecting an activity analysis algorithm relevant to the re-identification result (type of activity) so as to perform activity analysis, and sequentially storing the analysis result.

On the other hand, when the correctness (probability) of the identification result is between the first threshold and the second threshold, such as when a treadmill is being used and whether the detection target is walking or jogging cannot be judged, the correctness (probability) of the identification result is judged to be at a middle level. In this case, the control section 21 proceeds to Step B12 and activates the wireless communication section 26 so as to receive the beacon signal continuously transmitted from the electronic device 1. Here, the control section 21 judges whether the activity detection device 2 has entered the radio wave reachable range of the beacon signal, that is, whether the beacon signal has been normally received (Step B13). Then, when the beacon signal has not been normally received (NO at Step B13), the control section 21 returns to the above-described Step B2. Then, while identifying the type of the activity of the detection target by using the sensing motion signal, the control section 21 judges the correctness of the identification result (Steps B3 and B4) as described above, and repeats the above-described operations as long as the correctness is at the middle level.

During this repetition, when the beacon signal is normally received with the correctness (probability) of the identification result being at the middle level (YES at Step B13), the control section 21 acquires information (use object ID) characterizing the activity of the detection target from the advertising packet of the received beacon signal (Step 14), and then re-identifies the type of the activity of the detection target by using this acquired use object ID and the identification result acquired at the above-described Step B3 (Step B15). That is, the control section 21 re-identifies the type of the activity of the detection target by using the sensing motion signal (motion information) and the beacon signal (information characterizing the activity of the detection target). For example, in a case where plural types of activities have been taken as candidates by use of the sensing motion signal, if the number of the activity types is decreased by use of the beacon signal, the accuracy of identifying the type of the activity can be increased. By taking the re-identification result (type of activity) acquired thereby as effective, the control section 21 proceeds to the above-described Step B8 to Step B11 and performs processing of selecting an activity analysis algorithm relevant to the re-identification result (type of activity) so as to perform activity analysis, and sequentially storing the analysis result.

As described above, the control section 21 of the activity detection device 2 in the first embodiment acquires a sensing motion signal (motion information) that successively fluctuates in accordance with motions of a detection target as first information from the sensor section 25, acquires information characterizing the activity of the detection target as second information from the external electronic device 1, and identifies the type of the activity of the detection target by using these first information and second information. As a result of this configuration, various activities of a detection target can be appropriately detected for each type. That is, in the control section 21, even in the case of activity whose characteristic is inconspicuous in a sensing motion signal, the type of the activity can be easily detected without taking much time. Also, when the activity type of a detection target is changed, this change can be easily detected without taking much time. As such, various activities can be appropriately detected.

Also, the activity detection device 2 acquires information (use object ID) identifying an object being used by a detection target from the wireless communication section 26 of the electronic device 1 on the use object side. As a result of this configuration, by using this use object ID, activity that is being performed by a detection target can be accurately identified.

Moreover, the activity detection device 2 judges whether the activity of a detection target is detectable based on the above-described first information and, if it is detectable (if the correctness of the identification result is high), identifies the type of the activity of the detection target by use of the first information without using the above-described second information. If it is not detectable (if the correctness of the identification result is low), the activity detection device 2 identifies the type of the activity of the detection target by use of the second information. As a result of this configuration, the activity detection device 2 can accurately identify the type of activity by using the first information when the activity has a conspicuous characteristic or by using the second information when the activity has a less characteristic. As described above, the activity detection device 2 uses the second information conditionally. Therefore, it is not required to continuously detect the second information, which is advantageous in reduction in power consumption.

Furthermore, the activity detection device 2 determines whether to use the first information, the second information, or both of the first information and the second information based on the correctness of a detection result regarding the activity of a detection target, and thereby identifies the type of the activity of the detection target. As a result of this configuration, various activities of a detection target can be appropriately detected for each type, by different pieces of information being used such that identification when the correctness is high is performed by use of the first information, identification when the correctness is low is performed by use of the second information, and identification when the correctness is at a substantially middle level is performed by use of the first information and the second information. As such, when the correctness is at a substantially middle level, identification using the first information and the second information is performed. Accordingly, for example, whether a detection target at the time of using a treadmill is walking or jogging can be accurately identified.

Still further, the activity detection device 2 performs, on the first information, activity analysis corresponding to an identified type of activity. As a result of this configuration, various activities of a detection target can be appropriately detected for each type.

Yet still further, the activity detection device 2 selects an activity analysis algorithm corresponding to an identified type of activity from among the plurality of activity analysis algorithms prepared corresponding to plural types of activities in advance and analyzes the first information by following the selected activity analysis algorithm. As a result of this configuration, optimum analysis suitable for the activity of a detection target can be performed.

Yet still further, the activity detection device 2 acquires the second information by receiving a beacon signal transmitted from the electronic device 1. As a result of this configuration, the second information can be included in the advertising packet. Also, the short-distance wireless communication function of the existing Bluetooth (registered trademark) standards can be utilized as means for acquiring the second information.

Yet still further, the electronic device 1 transmits information characterizing the activity of a detection target. As a result of this configuration, the electronic device 1 can be used as an auxiliary device for the activity detection device 2. On the activity detection device 2 side, based on the transmitted information (characterization activity information), the type of the activity of the detection target can be easily identified.

Yet still further, in this activity detection system where the activity detection device 2 and the electronic device 1 have been communicably connected, the electronic device 1 transmits information characterizing the activity of a detection target. The activity detection device 2 acquires from the sensor section 25 a sensing motion signal (motion information) that successively fluctuates in accordance with motions of the detection target as first information, acquires from the external electronic device 1 the information characterizing the activity of the detection target as second information, and identifies the type of the activity of the detection target by using these first and second information. As a result of this configuration, various activities of a detection target can be appropriately detected for each type.

### First Modification Example

In the configuration of the above-described embodiment, the correctness (probability) of a result of identifying an activity type based on a sensing motion signal is judged, and the identification of the activity type and the activity analysis method are changed based on the judgment result. However, a configuration may be adopted in which the type of activity is always identified based only on a beacon signal transmitted from the electronic device 1. That is, a configuration may be adopted in which motion information that successively fluctuates in accordance with motions of a detection target is acquired as first information, information which is different from this motion information and characterizes the activity of the detection target is acquired as second information, and analysis corresponding to the type of activity identified by using the acquired second information is performed on the first information.

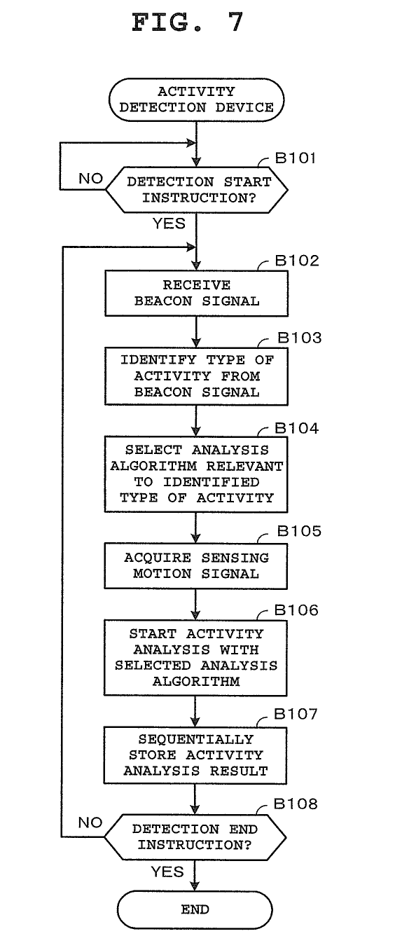

FIG. 7 is a flowchart for outlining the operation of the activity detection device 2 in a first modification example of the first embodiment.

First, when an instruction for starting detection is given (YES at Step B101), the control section 11 (more specifically, the CPU of the control section 11) of the activity detection device 2 receives a beacon signal from the electronic device 1 (Step B102), and identifies the type of activity by using only this beacon signal (Step B103). Subsequently, the control section 11 selects an analysis algorithm relevant to the identified activity type (Step B104), and then acquires a sensing motion signal from the sensor section 25 (Step B105). Next, the control section 11 starts activity analysis for analyzing the sensing motion signal by using the selected analysis algorithm (Step B106) and causes the analysis result to be sequentially stored in the storage section 23 (Step B107). Thereafter, the control section 11 returns to the above-described Step B102 (NO at Step B108), and repeats the above-described operations until an instruction for ending the detection is given.

By the type of the activity of the detection target being identified by using only the second information (beacon signal) as described above, the type of the activity can be more easily and reliably identified.

### Second Modification Example

In the configuration of the above-described first embodiment, when the activity detection device 2 is to re-identify the type of the activity of a detection target by using the first information and the second information, the type of the activity of the detection target is identified by an identification result (type of activity) using the first information and the second information acquired from the electronic device 1. However, a configuration may be adopted in which the activity detection device 2 identifies the type of the activity of a detection target by using the first information and the second information simultaneously. That is, a configuration may be adopted in which, without judging the correctness of the above-described identification result, the activity detection device 2 identifies the type of the activity of the detection target by using the first information and the second information simultaneously. By this configuration as well, various activities of a detection target can be appropriately detected for each type.

### Third Modification Example

In the above-described first embodiment, the use object ID that identifies a use object is described as “characterization activity information”. However, it may be an activity type ID that identifies activity or information that implies or suggests activity, as described above. Furthermore, the use object ID may be information (electronic device ID) that can identify the electronic device 1. That is, the electronic device 1 may be configured to transmit and output a beacon signal including the electronic device ID as “characterization activity information”. In this configuration, on the activity detection device 2 side, a correspondence table (omitted in the drawings) in which a plurality of electronic devices 1 and use objects are associated with one another is prepared, and searched based on an electronic device ID in a received beacon signal so as to identify a relevant use object. In this case, the contents of the correspondence table may be arbitrarily set by a user operation.

### Second Embodiment

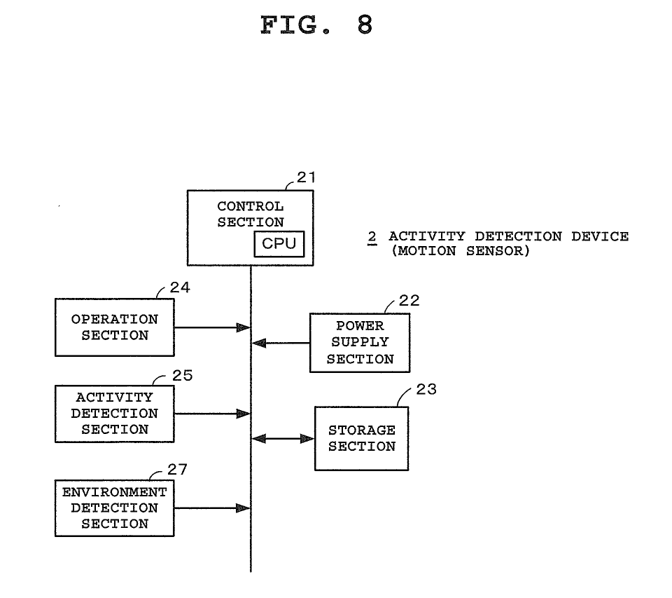

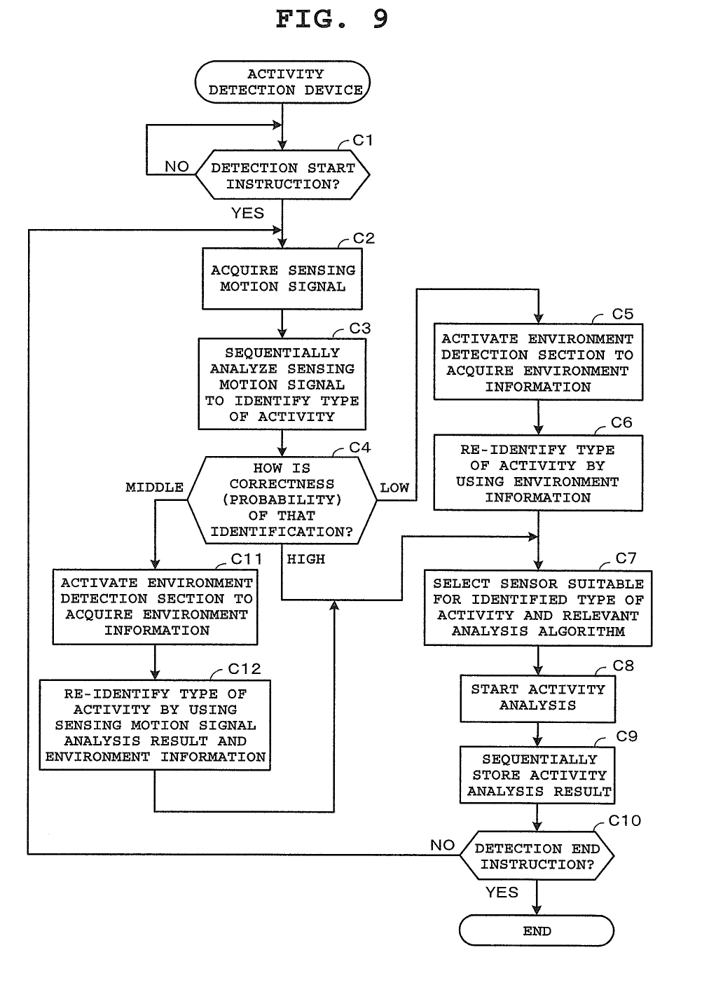

Next, a second embodiment of the present invention is described with reference to FIG. 8 and FIG. 9.

In the above-described first embodiment, the activity detection device 2 acquires, from the external electronic device 1, information characterizing the activity of a detection target as second information. In the second embodiment, the activity detection device 2 has an imaging function and a sound-collecting function, and acquires image information and sound information acquired by these functions as surrounding environment information. Here, sections that are basically the same as those of the first embodiment or sections having the same name in both embodiments are given the same reference numerals and descriptions thereof are omitted. Hereafter, the characteristic portions of the second embodiment are mainly described.

FIG. 8 is a block diagram showing basic components of the activity detection device 2 in the second embodiment.

As shown in FIG. 4, the activity detection device 2 has the control section 21 as its centerpiece. This activity detection device 2 of the second embodiment includes an environment detection section 27 in addition the power supply section 22, the storage section 23, the operation section 24, and the sensor section 25. As described above, the sensor section 25 is configured to have, for example, a triaxial acceleration sensor, gyro sensor, and geomagnetic sensor as various sensors to detect acceleration, inclination, direction and the like, and outputs a sensing motion signal that successively fluctuates in accordance with motions of a detection target.

The environment detection section 27 has a camera function and a sound-collecting function, and is configured to output the information of an image captured by a camera (omitted in the drawings) and sound information collected by a microphone (omitted in the drawings) as environment information regarding an environment surrounding a detection target. The control section 21 acquires a sensing motion signal (motion information) detected by the sensor section 25 and environment information detected by the environment detection section 27, and identifies the type of the activity of the detection target by using the motion information and the environment information. Note that, as with the first embodiment, “using the motion information and the environment information” is not limited to “using the motion information and the environment information simultaneously in time”, and there are specifications where the motion information and the environment information are used not simultaneously (for example, specifications in which a mode of using the first information and a mode of using the second information are provided and switching can be made between these modes) and specifications in which the motion information and the environment information are used simultaneously.

FIG. 9 is a flowchart outlining the operation of the activity detection device 2 in the second embodiment. Note that Steps C1 to C4 in FIG. 9 are similar processing corresponding to Steps B1 to B4 of FIG. 6, and Steps C7 to C10 are similar processing corresponding to Steps B8 to B11 of FIG. 6. Therefore, detailed descriptions of these steps are omitted herein.

When an instruction for starting detection is given (YES at Step C1), the control section 21 of the activity detection device 2 acquires a sensing motion signal (motion information) from the sensor section 25 (Step C2), identifies the type of the activity of the detection target by sequentially analyzing this sensing motion signal (Step C3), and judges the correctness (probability) of the identification result (Step C4). Then, when the correctness (probability) is high, the control section 21 takes the identified activity type as effective, and proceeds to Step C7 to Step C10 so as to perform processing of selecting an activity analysis algorithm relevant to the identification result (type of activity) and sequentially storing the analysis result.

When the correctness (probability) is low, the control section 21 proceeds to Step C5 and activates the environment detection section 27 so as to acquire surrounding environment information (image and sound) and re-identify the type of the activity of the detection target based on this environment information (Step C6). For example, when the detection target is performing an exercise such as skiing, playing golf, or playing baseball with the activity detection device 2 being attached to the front of his or her cap or an upper arm, the control section 21 re-identifies the type of the activity by using environment information indicating scenery, shouts, hitting sound, or the like as surrounding environment information. Then, the control section 21 takes the acquired re-identification result (type of activity) as effective, and proceeds to Step C7 to Step C10 to perform processing of selecting an activity analysis algorithm relevant to the re-identification result (type of activity) so as to perform activity analysis and sequentially storing the analysis result.

When the correctness (probability) of the identification result is at a middle level, the control section 21 activates the environment detection section 27 so as to acquire surrounding environment information (image and sound) (Step C11), and re-identifies the type of the activity of the detection target by using the acquired environment information and the identification result acquired at the above-described Step C3 (Step C12). That is, the control section 21 re-identifies the type of the activity of the detection target by using the sensing motion signal (motion information) and the environment information. Then, the control section 21 takes the acquired result of re-identification (type of activity) as effective, and proceeds to the above-described Step C7 to Step C10 to perform processing of selecting an activity analysis algorithm relevant to the re-identification result (type of activity) so as to perform activity analysis and sequentially storing the analysis result.

As described above, in the second embodiment, the control section 21 of the activity detection device 2 is configured to acquire motion information that successively fluctuates in accordance with motions of a detection target and identify the type of the activity of the detection target by using the acquired motion information and environment information. As a result of this configuration, various activities of a detection target can be appropriately detected for each type. For example, even when a characteristic is inconspicuous in a motion signal, the type of the activity can be easily detected without taking much time. Also, when the type of the activity of a detection target is changed, this change can be easily detected without taking much time. As such, various activities can be appropriately detected.

Also, in the above-described second embodiment, the environment detection section 27 is configured to have a camera function and a sound collection function. However, a configuration may be adopted in which the environment detection section 27 has an altimeter function, a GPS (Global Positioning System) detection function and the like so as to acquire environment information such as a current altitude, position and the like.

Moreover, in the above-described second embodiment, the activity detection device 2 has the environment detection section 27 incorporated therein. However, a configuration may be adopted in which the activity detection device 2 receives environment information from an external electronic device or a network.

Also, in the above-described embodiments, the activity detection device 2 is a device dedicated for activity detection. However, the present invention is not limited thereto, and any device having an activity detection function can be used as the activity detection device 2, such as a PDA (Personal Digital Assistant), a portable telephone such as a smartphone, an electronic game machine, a music player and the like.

Still further, the “devices” or the “sections” described in the above-described embodiments are not required to be in a single housing and may be separated into a plurality of housings by function. In addition, the steps in the above-described flowcharts are not required to be processed in time-series, and may be processed in parallel, or individually and independently.

While the present invention has been described with reference to the preferred embodiments, it is intended that the invention be not limited by any of the details of the description therein but includes all the embodiments which fall within the scope of the appended claims.

What is claimed is:

1. An activity detection device comprising:
a memory; and
a CPU,
wherein the CPU performs, based on a program stored in the memory, processing including:
acquiring motion information that successively fluctuates in accordance with motions of a detection target, as first information;
acquiring information characterizing activity of the detection target, as second information; and
identifying a type of the activity of the detection target by using the first information and the second information.

2. The activity detection device according to claim 1, wherein the CPU performs the processing including:
acquiring information which identifies a use object that is being used by the detection target or information which identifies the activity of the detection target as the second information, from an electronic device provided to the use object via a communication section.

3. The activity detection device according to claim 1, wherein the CPU performs the processing including:
judging whether the activity of the detection target is detectable based on the first information; and
identifying the type of the activity of the detection target by using the first information without using the second information when the activity is detectable, or identifying the type of activity of the detection target by using the second information when the activity is not detectable.

4. The activity detection device according to claim 1, wherein the CPU performs the processing including:
judging correctness of activity detection for the detection target based on the first information;
selecting one of a first method using the first information, a second method using the second information, and a third method using the first information and the second information, based on the correctness; and
identifying the type of the activity of the detection target by using a selected method.

5. The microphone according to claim 1, wherein the CPU performs the processing including:
identifying the type of the activity of the detection target by using the first information and the second information simultaneously.

6. The activity detection device according to claim 1, wherein the CPU performs the processing including:
performing analysis corresponding to the type of the activity on the first information.

7. The activity detection device according to claim 1, wherein the CPU performs the processing including:
selecting an activity analysis algorithm corresponding to the type of the activity from among a plurality of activity analysis algorithms prepared corresponding to plural types of activities in advance; and
analyzing the first information by following the selected activity analysis algorithm.

8. The activity detection device according to claim 1, wherein the CPU performs the processing including:
acquiring the second information by receiving a beacon signal transmitted from an electronic device.

9. The activity detection device according to claim 1, wherein the CPU performs the processing including:
acquiring information regarding an environment around the detection target as the second information.

10. An activity detection device comprising:
a memory; and
a CPU,
wherein the CPU performs, based on a program stored in the memory, processing including:
acquiring motion information that successively fluctuates in accordance with motions of a detection target, as first information;
acquiring information whose type is different from a type of the motion information and which is characterizing activity of the detection target, as second information;
identifying a type of the activity of the detection target by using the second information; and
performing analysis corresponding to the identified type of the activity of the detection target on the first information.

11. An activity detection system constituted by an activity detection device that detects activity of a detection target and an electronic device being communicably connected to each other, wherein the electronic device comprises a memory and a CPU which performs, based on a program stored in the memory, processing of transmitting information characterizing the activity of the detection target, and
wherein the activity detection device comprises a memory and a CPU which performs, based on a program stored in the memory, processing of (i) acquiring motion information that successively fluctuates in accordance with motions of the detection target, as first information, (ii) acquiring information characterizing the activity of the detection target, as second information, and (iii) identifying a type of the activity of the detection target by using the first information and the second information.

12. An activity detection method for an activity detection device comprising:
acquiring motion information that successively fluctuates in accordance with motions of a detection target, as first information;
acquiring information characterizing activity of the detection target, as second information; and
identifying a type of the activity of the detection target by using the first information and the second information.
